(19) United States
(12) Patent Application Publication
(10) Pub. No.: US 2013/0115180 A1
(43) Pub. Date: May 9, 2013

(54) Methods And Compositions For Administering A Specific Wavelength Phototherapy

(71) Applicants: Andy Ofer Goren, Newport Beach, CA (US); John McCoy, Downey, CA (US)

(72) Inventors: Andy Ofer Goren, Newport Beach, CA (US); John McCoy, Downey, CA (US)

(21) Appl. No.: 13/669,435

(22) Filed: Nov. 5, 2012

Related U.S. Application Data
(60) Provisional application No. 61/555,130, filed on Nov. 3, 2011.

(51) Int. Cl. A61K 8/60; A61K 31/7034; A01G 1/00; A61K 8/40; A61Q 17/04; B01J 19/12; A61N 5/06; A61K 31/24

(52) U.S. Cl. CPC A61K 8/602; A61N 5/062; A61K 31/7034; A61K 31/24; A61K 8/40; A61Q 17/04; B01J 19/12; A01G 1/001
USPC 424/60; 604/20; 250/492.1; 250/472.1; 47/58.1 LS

(57) ABSTRACT

Methods and compositions are disclosed for administering electromagnetic radiation (EMR), for therapeutic or cosmetic purposes, or for purposes of curing a polymeric material.

### CROSS-REFERENCE TO RELATED APPLICATIONS

This application claims priority to U.S. Provisional Application No. 61/555,130, filed Nov. 3, 2011, entitled “System and Method for Administering a Specific Wavelength Phototherapy,” which is incorporated herein in its entirety.

### BACKGROUND

1. Technical Field

The inventions described herein relate to methods and compositions for administering electromagnetic radiation (EMR), for therapeutic or cosmetic purposes, or for purposes of curing a polymeric material.

2. Description of the Related Art

Ultraviolet (UV) phototherapy is a well-established treatment for several types of dermatological disease. It is commonly administered to treat psoriasis, vitiligo, atopic dermatitis, and other skin conditions. Studies have shown that narrow-band ultraviolet B (NB-UVB) phototherapy is a safer and more effective alternative to other UV phototherapies, including broad-band UVB and oral psoralin with UVA (PUVA) treatment. For example, psoriasis studies have established that NB-UV light in the range 310-315 nm has the best therapeutic benefit with the least potential side effects. Typical treatments use narrow band UV-B with maximum wavelength intensity centered at 311 nm as a practical consequence of the availability of NB-UVB light sources.

Phototherapy is typically administered in medical offices and depending on the condition and the individual being treated may require a significant time and financial commitment. For example, vitiligo patients undergoing NB-UVB phototherapy typically visit the medical office two to three times per week for a period of two to three months to have beneficial results. The significant time commitment and associated cost is the main drawback to NB-UVB phototherapy. Therefore, an NB-UVB phototherapy alternative that can be safely applied and controlled by patients would be beneficial.

Portable phototherapy lamps are available for in home use; however, applying the proper and effective dosage may be difficult and unsafe for patients. In addition, when phototherapy is administered at medical offices, an artificial light source (NB-UVB) is used. The light source emits NB-UVB at a specific therapeutic range as well as a significant amount of non-therapeutic harmful UVB. A topical agent that can reduce harmful radiation exposure at the clinic will be highly valuable for patient safety.

Vitamin D is an essential nutrient for human health that promotes the growth of bone. Vitamin D is acquired by humans in diet or endogenously synthesized with adequate sun exposure. Not all wavelengths of light promote the synthesis of vitamin D equally. Similarly, the erythema (sunburn) reaction of skin is also wavelength dependent.

Research has indicated that UV-B light in the range 306-310 nm has the greatest offset of benefit for the production of vitamin D versus the negative effects of erythema. As such, a band-pass therapeutic cream that selectively passes radiation in this region would be an improvement to currently available sunscreens, which completely inhibit the endogenous synthesis of vitamin D from sun exposure.

Additionally, UV light sources are commonly used in the manufacturing industry for drying inks, coatings, adhesives and other UV sensitive materials through polymerization (curing). Selecting the right spectral output is vital for UV-curing performance. Unfortunately, UV-curing radiation sources often emit a broad spectrum of UV radiation that may contain wavelengths of light that are not beneficial to the curing process but may produce negative effects in the manufactured product (e.g. heating). As such, a UV radiation band-pass filter that could selectively pass desirable wavelengths of light would be beneficial to the use of curing in manufacturing processes.

### BRIEF SUMMARY

Described herein are methods for administering a specific wavelength of electromagnetic radiation while excluding electromagnetic radiation of other frequencies for biological purposes in living organisms including medical therapy, health supportive therapy, health maintenance, cosmetic desire, vitamin production or other reasons. In addition, methods are described for applying a specific wavelength of electromagnetic radiation to an object for the purpose of curing in a manufacturing process.

One embodiment described herein is a method delivering a dose of electromagnetic radiation (EMR) to an object, comprising the steps of: covering the object with a composition which selectively allows passage of EMR of one or more predetermined wavelengths, while excluding other wavelengths; and exposing said object to a light source that includes EMR of a spectrum that includes said one or more predetermined wavelengths. Preferably, the object is the skin of a human subject, the composition is a band-pass topical photocream, the step of covering the object comprises application of said photocream, and the one or more predetermined wavelengths are ultraviolet wavelengths selected to provide phototherapy to the subject, and wherein the step of exposing comprises exposing the skin to sunlight or an artificial ultraviolet light source.

Another embodiment described herein is a composition comprising: a cosmetic-grade carrier lotion suitable for application to human skin; and a first component and a second component, each selected from the group consisting of hesperidin (CAS #520-26-3), vinblastine (CAS #865-21-4), acteoside (CAS #61276-17-3), acacetin 7-0-rutinoside (CAS #480-36-4), phytoene (CAS #13920-14-4), poncirin (CAS #14941-08-3), gambogic acid (CAS #2752-65-0), chaetoglobosin (CAS #50335-03-0), poliumoside (CAS #94079-81-9), sitosteroline (CAS #474-58-8), naringin (CAS#10236-47-2), pentagalloyl glucose (CAS #14937-32-7), amentoflavone (CAS #1617-53-4), tetrandrine (CAS #518-34-3), isoacteoside (CAS #61303-13-7), (−)-phaeanthine (CAS #1263-79-2), garcinol (CAS #78824-30-3), salvianolic acid B (CAS #121521-90-2), docetaxel (CAS #114977-28-5), ecdysterone (CAS #5289-74-7), glycyrrhizic acid monosodium salt (CAS #11052-19-0), kaempferol (CAS #81992-85-0), paclitaxel (CAS #33069-62-4), silymarin (CAS #22888-70-6), isoacteoside (CAS #61303-13-7), linarin (CAS #480-36-4), pectolinarin (CAS #28978-02-1), rutin (CAS #153-18-4), kaempferol-3-O-rutinoside (CAS #17650-84-9), diosmin (CAS #520-27-4), rhoifolin (CAS #17306-46-6), avobenzone (CAS #70356-09-1), alpha glucosyl hesperidin (CAS #161713-86-6), and diethylamino hydroxybenzoyl hexyl benzoate (CAS #302776-68-7), wherein the first component and the second component are selected, and included within the composition at a mutual ratio so as to produce an absorption spectrum with a valley at approximately 306 nm to approximately 320 nm, and wherein the concentration of the first component and the second component in the carrier lotion is between about 0.10% (w/w) and about 5% (w/w).

Other embodiments are disclosed herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings, where like reference numerals refer to like reference in the specification:

FIG. 1 is a flowchart showing a method of applying a photocream.

FIG. 2 shows an example of a computerized system for conducting or analyzing an assay to test DNA samples and providing a result.

FIG. 3 is an example of absorption spectra of photocream containing 0.75% (w/w) Silymarin (CAS #22888-70-6) and 1.125% (w/w) diethylamino hydroxybenzoyl hexyl benzoate (CAS #302776-68-7) applied at a thickness of 20 μm

FIG. 4 shows a transmittance profile for a band-pass photocream.

FIG. 5 is a representation of an example of wavelength dependent erythema weighted irradiance.

FIG. 6 shows an example UV transmittance spectrum of a photocream formulated with 2% (w/w) Silymarin (CAS #22888-70-6), when applied at a thickness of 20 μam

FIG. 7 shows an example absorption spectrum of a photocream containing 1% (w/w) Silymarin (CAS #22888-70-6) and 2.5% (w/w) diethylamino hydroxybenzoyl hexyl benzoate (CAS #302776-68-7), when applied at a thickness of 20 μm

FIG. 8 shows a transmittance profile for a band-pass photocream determined from the UV absorption spectrum of FIG. 7.

### DETAILED DESCRIPTION

The description herein is provided in the context of a system and method for administering a phototherapy. Those of ordinary skill in the art will realize that the following detailed description is illustrative only and is not intended to be in anyway limiting. Other embodiments will readily suggest themselves to such skilled persons having the benefit of this disclosure. Reference will now be made in detail to implementations as illustrated in the accompanying drawings. The same reference indicators will be used throughout the drawings and the following detailed description to refer to the same or like parts.

In the interest of clarity, not all of the routine features of the implementations described herein are shown and described. In the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In one embodiment disclosed herein, a band-pass photocream is used to selectively filter radiation in the UVB region of the electromagnetic spectrum. The chemical composition of the photocream may be such that it absorbs wavelengths of light that are non-beneficial to the treatment of the aforementioned skin ailments. Simultaneously, the band-pass cream may selectively pass wavelengths of radiation that are beneficial for treatment. Application of the photocream is followed by exposure to either natural (sun) or artificial light. In various alternative embodiments, the filtering mechanism can be in the form of a topical agent, a film, an article of clothing, a lens, a window glass, or other light filtration mechanism having an equivalent effect.

After application of the photo-filtration device, a person (or other biological organism) could receive a controlled dose of phototherapy throughout the day. This would greatly reduce the inconvenience of the standard method of delivering phototherapy in medical offices. Furthermore, the band-pass photocream could be formulated into different dosages depending on the required amount of phototherapy, physiology, genetics of the user or the condition being treated.

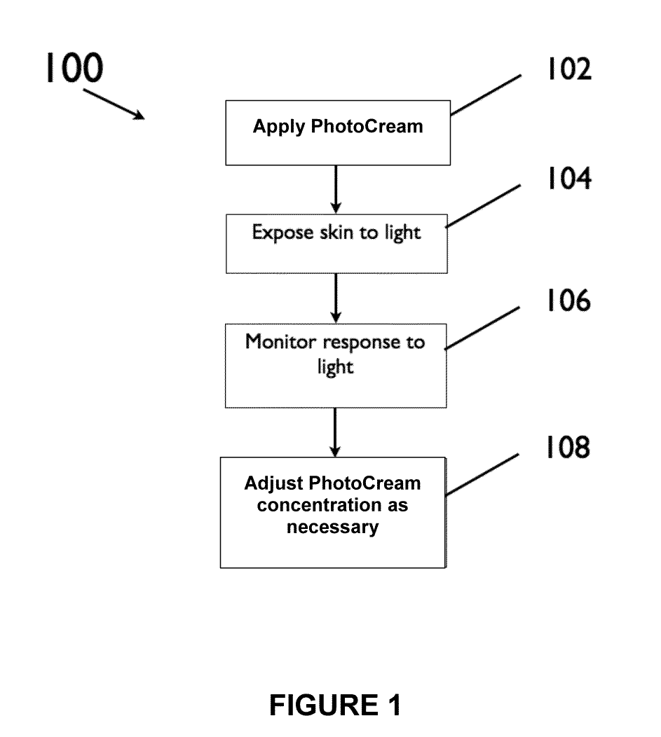

With reference to FIG. 1, a method 100 is illustrated. A band-pass photocream may be applied (102) to an exposed skin surfaces requiring phototherapy. Then, the skin surfaces may be exposed (104) to light, either as natural (sun) or artificial light. The dosage of therapeutic radiation received at the skin may be monitored (106), by the user, other personnel, or by a monitoring device such as an image-based electronic device, radiation absorption device or other method. A dosimeteter device may in one embodiment measure both therapeutic radiation and non-therapeutic radiation, or either of them separately. Furthermore, a wearable device in the form of an adhesive UV dosimeter appliqué could be used to monitor the amount of radiation exposure a person has received. The UV dosimeter appliqué could be applied to the skin prior to addition of the band-pass photocream and would itself be treated with the photocream; in another embodiment, the UV dosimeter appliqué could be treated with a polymer coating containing the same or similar (having closely related UV absorption) chemical actives as the band-pass photocream. Photocream concentration may then be adjusted (108) as necessary.

Delivery of UV light may be provided by sunlight, a UV lamp, a fluorescent tube, through amplification of available light such as through a fluorescence energy transfer reaction (FRET), or chemical, molecular, or other approaches known in the art.

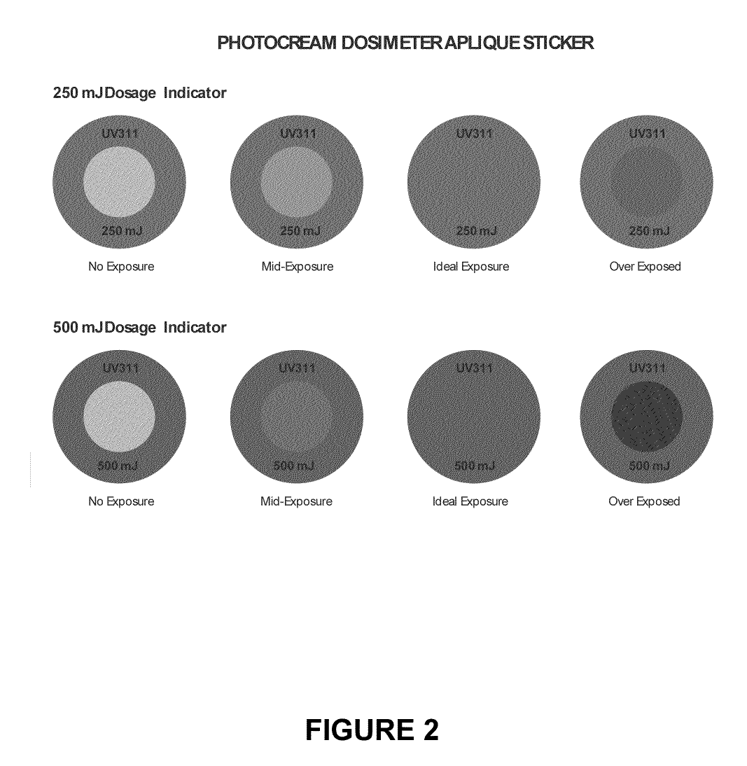

FIG. 2 illustrates an embodiment of a UV dosimeter appliqué. Two halves of a geometric shape may be used to report proper dosage of therapeutic UVB exposure. In one half of the geometric shape, a UV reactive dye may be printed. The chemistry of the dye may be such that the dye will change color in a UV dosage dependent manner. The color change of the dye may be calibrated, empirically, in a controlled laboratory environment by exposing the printed dye to a known amount of UV radiation. The empirically observed color may then be printed with standard dyes (non-UV reactive) onto the outer half of the geometric shape. This arrangement would allow for ease of use by the user in correlating color change with proper UV dosage. The UV dosimeter appliqué may be replaced with a similar device, such as a wrist band, ring or a watch.

In another embodiment of the UV dosimeter appliqué, two or more UV-reactive inks may be used to create a dosimeter that reports exposure to different bands of UV radiation. Each UV-reactive ink may have chemistry such that each ink would absorb UV radiation at separate bands (i.e. would change color based on the absorption of UV radiation at different wavelengths). As such, the system could be used to monitor exposure to UV radiation that would be considered therapeutic for a particular skin condition versus radiation that would be considered non-therapeutic. Alternatively, a therapeutic versus non-therapeutic determining dosimeter could be constructed using a broad-band UV absorbing dye that is treated with different polymer coatings containing UV absorbing actives that would filter out either therapeutic or non-therapeutic UV. The dosimeter is not limited to a chemical dosimeter, but could in one of several embodiments employ an electronic photosensor.

In yet another embodiment, a photoactive molecule may be added to the photocream; said molecule may change its chemical structure after a threshold level of UV exposure such that it would become opaque to UV radiation after receiving an appropriate dosage. As such, the added molecule would protect (block) the user from further exposure. This may be a manner in which, according to FIG. 1, the band-pass photocream concentration is adjusted (108) as required for optimum treatment benefits. The adjustments can be made based upon a database of patient conditions, treatment response, physiology, or genetics of the user and state of a device as described above in 106 or other input and/or computer analysis.

It may also be possible to use a computed analysis to select the optimum band-pass photocream concentration and/or light dosage based on the patient's response to a given concentration of the photocream with or without other characteristics of physiology or genetics of the user. According to such an approach, a method for predicting optimum photocream concentration may include: (a) constructing a N-layer neural network; (b) training the neural network with a data set of patients who have characteristics that relate to response to the photocream for the treatment of dermatological conditions, such as vitiligo, psoriasis, atopic dermatitis, etc.; (c) obtaining an image of skin response from the subject, including concentration of the photocream and light dosage; (d) generating a response-based profile from the sample, the profile being a function of values associated with a prescribed set of phototherapy parameters; (e) obtaining a difference vector from the profile; (f) inputting the difference vector into the neural network. The necessary patient data may be able to be collected from a personal device and automatically supply real time monitoring and adjustments.

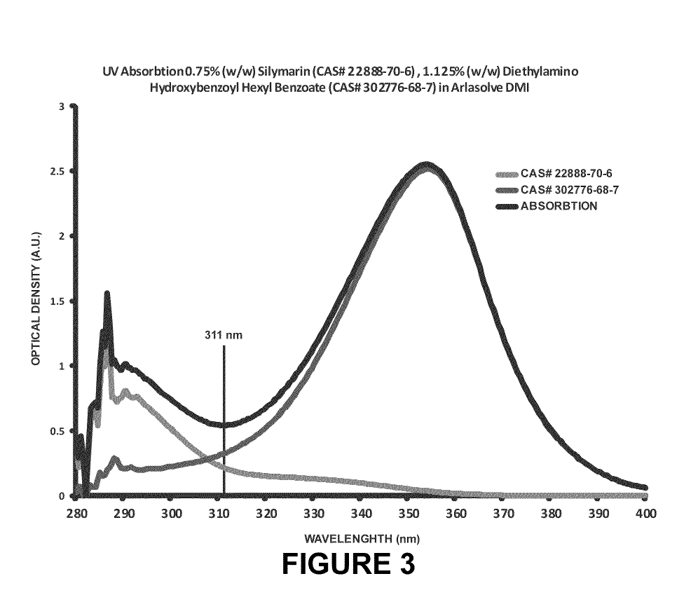

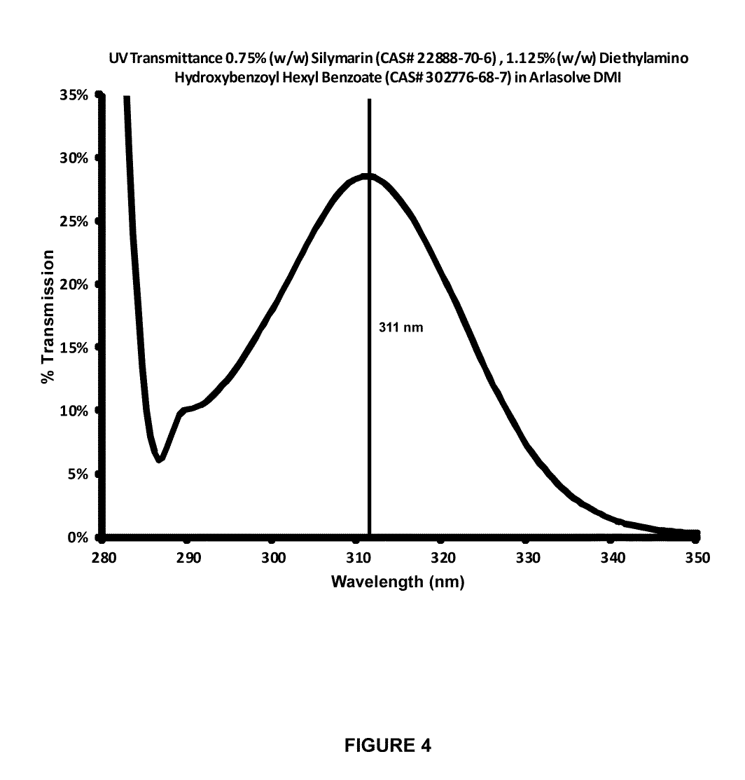

In one embodiment of the present invention, a band-pass photocream is composed such that it is optimized to have maximum transmittance at a therapeutic wavelength of 311 nm for the treatment of vitiligo, psoriasis, atopic dermatitis, and other skin conditions. Said photocream would contain two UV absorbing active ingredients having UV absorption spectra that when combined in a determined ratio would have a spectral minimum (valley) at 311 nm. For example, a band-pass photocream could be formulated with Silymarin (CAS #22888-70-6) and diethylamino hydroxybenzoyl hexyl benzoate (CAS #302776-68-7) in a weight to weight ratio of 2:3 (or less preferably within the range 1:2 to 5:6, or within the range 5:9 to 7:9) to produce an absorption spectrum with a spectral valley at 311 nm. Said photocream may contain 0.75% (w/w) Silymarin (CAS #22888-70-6) and 1.125% (w/w) diethylamino hydroxybenzoyl hexyl benzoate (CAS #302776-68-7). An illustrative absorption spectra for such a composition is shown in FIG. 3 when applied at a thickness of 20 μm. From the UV absorption spectrum in FIG. 3, a transmittance profile for a band-pass photocream may be determined as illustrated in FIG. 4, which in this example indicates a maximum transmittance (about 29%) at 311 nm. Alternatively, a band-pass photocream could be formulated with alpha glucosyl hesperidin (CAS #161713-86-6) and diethylamino hydroxybenzoyl hexyl benzoate (CAS #302776-68-7) in the weight to weight ratio of 4:1 (or less preferably within the range 3:1 to 5:1, or within the range 7:2 to 9:2) to produce an absorption spectrum with a spectral valley at 311 nm.

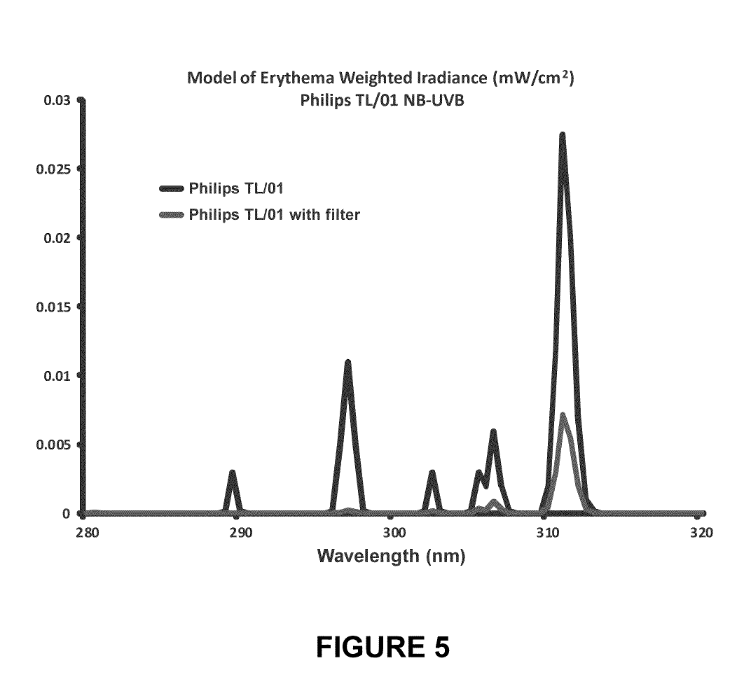

Typical light sources for the treatment of vitiligo have been reported to deliver approximately 66% of their erythema weighted irradiance in the therapeutic range 310-320nm. The remaining erythema weighted irradiance (34%) may be delivered at wavelengths below 310 nm, which can have negative health consequence for users (e.g. erthema and cancer). An example representing the wavelength dependent erythema weighted irradiance is shown in FIG. 5.

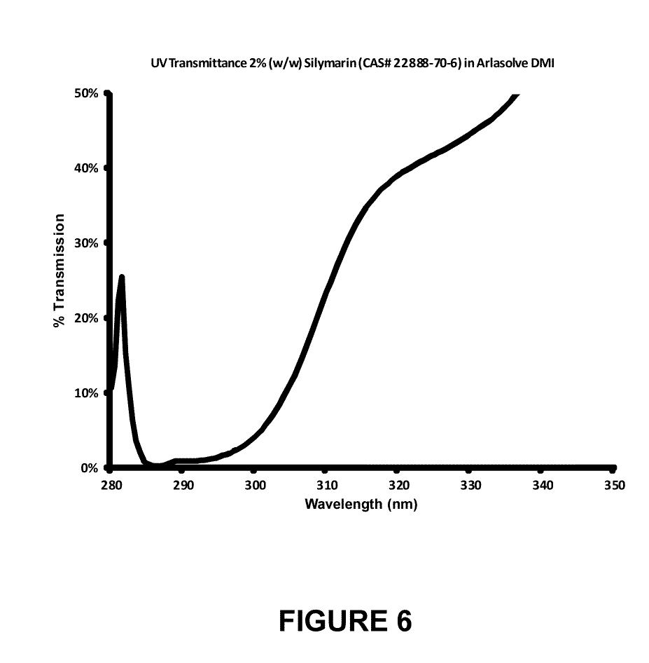

In another embodiment, a combination of UV absorbing molecules may be formulated to selectively filter non-therapeutic wavelengths of light from an artificial light source. The filtering mechanism can be in the form of a topical agent, a film, an article of clothing, a lens, or other light filtration mechanism having an equivalent effect. For example, a photocream may be formulated with 2% (w/w) Silymarin (CAS #22888-70-6) and might produce the UV transmittance spectrum in FIG. 6 when applied at a thickness of 20 μm. From the UV transmittance spectrum in FIG. 6, an adjusted erythema weighted irradiance of the Phillips TL01 (FIG. 5) may be calculated, and in this example predicts delivery of 87% of the erythema weighted irradiance in the therapeutic range 310-320 nm.

The above exemplary mode of carrying out the invention is not intended to be limiting as other methods of initiating a filter between the radiation source and radiation destination are possible. For example, a similar chemistry to the photocream described above can be incorporated into a polymer coating and applied directly to a fluorescent tube or embedded in a screen placed between the radiation source and the intended radiation destination.

In one embodiment, a band-pass therapeutic cream that selectively passes radiation in the region of UV-B light in the range 306-310 nm. This region has the greatest offset of benefit for the production of vitamin D versus the negative effects of erythema. Therefore, this embodiment would provide limited protection from the deleterious effects of sun exposure (erthema) while still allowing natural synthesis of vitamin D in skin.

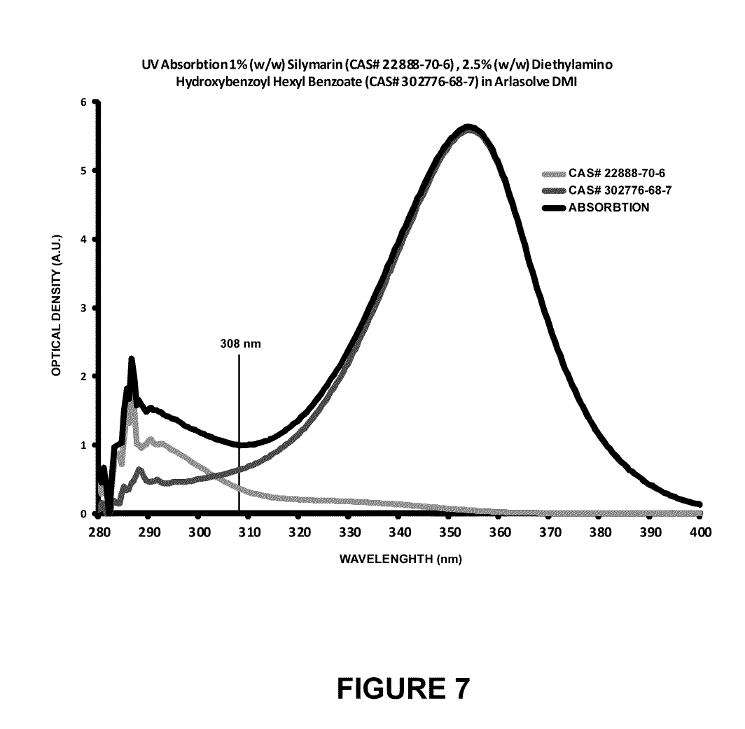

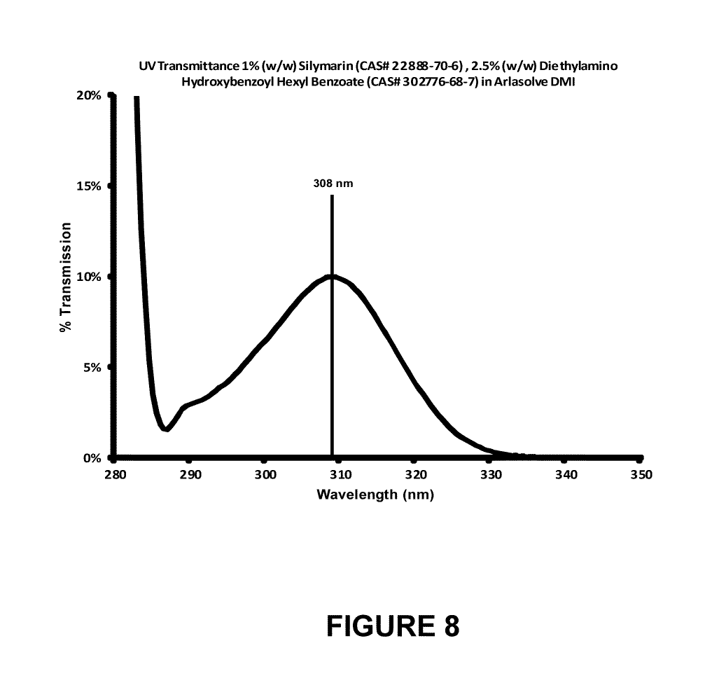

In yet another embodiment, a combination of UV absorbing molecules may be formulated to selectively pass UV-B light in the range 306-310 nm for the benefit of maximum vitamin D production while still providing limited protecting from erythema. Said photocream may contain two UV absorbing active ingredients having UV absorption spectra that when combined in a determined ratio would have a spectral minimum (valley) at 308 nm. For example, a band-pass photocream could be formulated with Silymarin (CAS #22888-70-6) and diethylamino hydroxybenzoyl hexyl benzoate (CAS #302776-68-7) in a weight to weight ratio of 2:5 (or less preferably within the range 3:10 to 1:2, or within the range 1:3 to 7:15) to produce an absorption spectrum with a spectral valley at 308 nm. Said photocream could contain 1% (w/w) Silymarin (CAS #22888-70-6) and 2.5% (w/w) diethylamino hydroxybenzoyl hexyl benzoate (CAS #302776-68-7) and might produce the absorption spectra such as that shown in FIG. 7 when applied at a thickness of 20 μm. From the UV absorption spectrum in FIG. 7, a transmittance profile for a band-pass photocream can be determined as exemplified in FIG. 8, which in this example indicates a maximum transmittance (about 10%) at 308 nm. Alternatively, a band-pass photocream could be formulated with alpha glucosyl hesperidin (CAS #161713-86-6) and diethylamino hydroxybenzoyl hexyl benzoate (CAS #302776-68-7) in a weight to weight ratio of 3:2 (less preferably a range of 5:4 to 7:4, or a range of 4:3 to 5:3) to produce an absorption spectrum with a spectral valley at 308 nm.

UV light sources are commonly used in the manufacturing industry for drying inks, coatings, adhesives and other UV sensitive materials through polymerization (curing) in lieu of evaporation. Selecting the right spectral output is vital for UV-curing performance. In general, UV-cured materials do not react the same way to UV radiation, but instead have selective responses to wavelength variations. Unfortunately, UV-curing radiation sources often emit a broad spectrum of UV radiation that may contain wavelengths of light that are not beneficial to the curing process but may produce negative effects in the manufactured product (e.g. heating). As such, a UV radiation band-pass filter that could selectively pass desirable wavelengths of light would be beneficial to the use of curing in manufacturing processes.

In yet another embodiment, a UV absorbing molecule or a combination of UV absorbing molecules may be formulated to selectively pass UV light that is most beneficial to a particular curing agent (e.g. a dye). The UV absorbing or reflective molecules could be embedded or doped into a polymeric sheet or painted on a quartz pane. These sheets may constitute a selective wavelength filter and could be used alone or combined (stacked) to achieve an appropriate band-pass filter for UV radiation. The filter may then be placed between the radiation source and the intended radiation destination. The above exemplary mode of carrying out the invention is not intended to be limiting as other methods of initiating a filter between the radiation source and radiation destination are possible. For example, a similar chemistry could be incorporated into a gel and applied directly to the intended radiation destination or the chemistry could be incorporated into a transparent mold that would benefit curing of parts normally inaccessible to light (i.e. the bottom of the mold).

Other combinations of UV absorbing actives are possible to achieve similar results to those described in the above disclosures. Examples of comparable UV absorbing active include but are not limited to: hesperidin (CAS #520-26-3), vinblastine (CAS #865-21-4), acteoside (CAS #61276-17-3), acacetin 7-O-rutinoside (CAS #480-36-4), phytoene (CAS #13920-14-4), poncirin (CAS #14941-08-3), gambogic acid (CAS #2752-65-0), chaetoglobosin (CAS #50335-03-0), poliumoside (CAS #94079-81-9), sitosteroline (CAS #474-58-8), naringin (CAS #10236-47-2), pentagalloyl glucose (CAS #14937-32-7), amentoflavone (CAS #1617-53-4), tetrandrine (CAS #518-34-3), isoacteoside (CAS #61303-13-7), (-)-phaeanthine (CAS #1263-79-2), garcinol (CAS #78824-30-3), salvianolic acid B (CAS #121521-90-2), docetaxel (CAS #114977-28-5), ecdysterone (CAS #5289-74-7), glycyrrhizic acid monosodium salt (CAS #11052-19-0), kaempferol (CAS #81992-85-0), paclitaxel (CAS #33069-62-4), silymarin (CAS #22888-70-6), isoacteoside (CAS #61303-13-7), linarin (CAS #480-36-4), pectolinarin (CAS #28978-02-1), rutin (CAS #153-18-4), kaempferol-3-O-rutinoside (CAS #17650-84-9), diosmin (CAS #520-27-4), rhoifolin (CAS #17306-46-6), avobenzone (CAS #70356-09-1), alpha glucosyl hesperidin (CAS #161713-86-6), mycosporine-like amino acids, rare earth metals. Variants of these components may also be used, as well as other substances known to absorb EMR, and preferably ultraviolet light.

Alternatively, a molecule may be selected such that its absorbance maximum corresponds to the wavelength of the most therapeutic value; said molecule could then be synthesized such that a conjugated bond may be added to the molecule; in addition a second molecule would be synthesized such that a conjugated bond would be subtracted from the original molecule. In each of the synthesis schemes described above the absorption maxima of the molecule would be red-shifted or blue-shifted accordingly (i.e. increased in wavelength or decreased in wavelength). As such, an equal molar combination of the molecules would produce a filter with an absorption minimum (“valley”) at the wavelength of the absorption maximum of the original molecule.

The above are exemplary modes of carrying out the invention and are not intended to be limiting. It will be apparent to those of ordinary skill in the art that modifications thereto can be made without departure from the spirit and scope of the invention as set forth in the following claims.

What is claimed is:

1. A method delivering a dose of electromagnetic radiation (EMR) to an object, comprising the steps of:
covering the object with a composition which selectively allows passage of EMR of one or more predetermined wavelengths, while excluding other wavelengths; and
exposing said object to a light source that includes EMR of a spectrum that includes said one or more predetermined wavelengths.

2. The method of claim 1, further comprising the step of stopping the exposure of the object to the light source when the object has received a predetermined amount of EMR at the one or more predetermined wavelengths.

3. The method of claim 2, wherein said step of stopping occurs after a predetermined time after the beginning of said step of exposure, further comprising the step of adjusting the concentration of said composition within a carrier material so as to only allow, within the predetermined time, said predetermined amount of EMR.

4. The method of claim 2, further comprising the step of providing a dosimeter that measures exposure to at least one of said one or more predetermined wavelengths, and that provides an indication when said object has received a predetermined amount of EMR at the measured wavelength or wavelengths; wherein the step of stopping the exposure occurs when the dosimeter provides said indication.

5. The method of claim 4, further comprising the step of covering the dosimeter with said composition.

6. The method of claim 2, wherein said composition comprises a photoactive molecule that changes its chemical structure after being exposed to a threshold level of EMR, such that it becomes opaque to at least one of the one or more predetermined wavelengths; and wherein the step of stopping the exposure occurs when said photoactive molecule thus changes its chemical structure.

7. The method of claim 1, wherein one of said one or more predetermined wavelengths is approximately 311 nm.

8. The method of claim 1, wherein one of said one or more predetermined wavelengths is approximately 308 nm.

9. The method of claim 1, wherein said composition comprises a first composition that preferentially blocks EMR below one of said one or more predetermined wavelengths, and a second composition that preferentially blocks EMR above said one of said one or more predetermined wavelengths.

10. The method of claim 1, wherein said composition comprises one or more active photocream ingredients selected from the group consisting of avobenzone, cinoxate, dioxybenzone, Ecamsule, ensulizole, Escalol 507, Escalol 567, Eusolex 4360, Eusolex 6007, Eusolex 6300, Eusolex 8020, homosalate, meradimate, Mexenon, octocrylene, octinoxate, octisalate, oxybenzone, padimate 0, Parsol 1789, Parsol MCX, sulisobenzone, and trolamine salicylate.

11. The method of claim 1, wherein said composition comprises one or more components selected from the group consisting of scytonemin, mycosporine, mycosporine-glycine:valine, mycosporine-glycine, anthocyanidin, asterina, dihydroflavonol, flavone, flavanol, gadusol, isoflavone, nostoc commune E335, palythene, palythenic acid, palythine, palythinol, porphyra, and shinorine.

12. The method of claim 1, wherein said step of exposing comprises delivering of light that has been amplified at one or more particular wavelengths.

13. The method of claim 12, wherein said amplification comprises fluorescence emission, and said one or more particular wavelengths are said one or more predefined wavelengths.

14. The method of claim 1, wherein the object is a plant sensitive to at least one of said one or more particular wavelengths.

15. The method of claim 1, wherein the object is a polymer which is capable of being cured by exposure to EMR at said one or more predetermined wavelengths.

16. The method of claim 1, wherein the object is the skin of a human subject, the composition is a band-pass topical photocream, the step of covering the object comprises application of said photocream, and the one or more predetermined wavelengths are ultraviolet wavelengths selected to provide phototherapy to the subject, and wherein the step of exposing comprises exposing the skin to sunlight or an artificial ultraviolet light source.

17. The method of claim 16, wherein the phototherapy is for treatment of a dermatological condition selected from the group consisting of vitiligo, psoriasis, atopic dermatitis, grey hair, and skin infection.

18. The method of claim 16, wherein the phototherapy is for wound healing, varicose vein therapy, androgenetic alopecia, or other forms of alopecia.

19. The method of claim 16, wherein the phototherapy is for treatment of rickets, osteomalacia, osteoporosis, other forms of impaired bone mineralization, or low blood calcidiol.

20. A composition comprising:
a cosmetic-grade carrier lotion suitable for application to human skin; and
a first composition, suitable for application to human skin, that preferentially blocks EMR below one of said one or more predetermined wavelengths, and a second composition, suitable for application to human skin, that preferentially blocks EMR above said one of said one or more predetermined wavelengths, wherein the first component and the second component are selected, and included within the composition at a mutual ratio so as to produce an absorption spectrum with a valley at approximately 306 nm to approximately 320 nm, and wherein the concentration of the first component and the second component in the carrier lotion is between about 0.10% (w/w) and about 5% (w/w).

21. The composition of claim 20, wherein said valley is between about 306 nm and about 310 nm.

22. The composition of claim 21, wherein said valley is approximately 308 nm.

23. The composition of claim 20, wherein said valley is approximately 311 nm.

24. The composition of claim 20, wherein a first component and a second component, are each selected from the group consisting of hesperidin (CAS #520-26-3), vinblastine (CAS #865-21-4), acteoside (CAS #61276-17-3), acacetin 7-O-rutinoside (CAS #480-36-4), phytoene (CAS #13920-14-4), poncirin (CAS #14941-08-3), gambogic acid (CAS #2752-65-0), chaetoglobosin (CAS #50335-03-0), poliumoside (CAS #94079-81-9), sitosteroline (CAS #474-58-8), naringin (CAS #10236-47-2), pentagalloyl glucose (CAS #14937-32-7), amentoflavone (CAS #1617-53-4), tetrandrine (CAS #518-34-3), isoacteoside (CAS #61303-13-7), (-)-phaeanthine (CAS #1263-79-2), garcinol (CAS #78824-30-3), salvianolic acid B (CAS #121521-90-2), docetaxel (CAS #114977-28-5), ecdysterone (CAS #5289-74-7), glycyrrhizic acid monosodium salt (CAS #11052-19-0), kaempferol (CAS #81992-85-0), paclitaxel (CAS #33069-62-4), silymarin (CAS #22888-70-6), isoacteoside (CAS #61303-13-7), linarin (CAS #480-36-4), pectolinarin (CAS #28978-02-1), rutin (CAS #153-18-4), kaempferol-3-O-rutinoside (CAS #17650-84-9), diosmin (CAS #520-27-4), rhoifolin (CAS #17306-46-6), avobenzone (CAS #70356-09-1), alpha glucosyl hesperidin (CAS #161713-86-6), and diethylamino hydroxybenzoyl hexyl benzoate (CAS #302776-68-7), wherein the first component and the second component are selected, and included within the composition at a mutual ratio so as to produce an absorption spectrum with a valley at approximately 306 nm to approximately 320 nm, and wherein the concentration of the first component and the second component in the carrier lotion is between about 0.10% (w/w) and about 5% (w/w).

25. The composition of claim 24, wherein the first component is alpha glucosyl hesperidin and the second component is diethylamino hydroxybenzoyl hexyl benzoate.

26. The composition of claim 25, wherein said mutual ratio is between about 7:2 and about 9:2 of the first component to the second component.

27. The composition of claim 26, wherein said mutual ratio is about 4:1 of the first component to the second component.

28. The composition of claim 25, wherein said mutual ratio is between about 4:3 and about 5:3 of the first component to the second component.

29. The composition of claim 28, wherein said mutual ratio is about 3:2 of the first component to the second component.

30. The composition of claim 24, wherein the first component is Silymarin and the second component is diethylamino hydroxybenzoyl hexyl benzoate.

31. The composition of claim 30, wherein said mutual ratio of the first component to the second component is within a range selected from the group consisting of about 5:9 and about 7:9 and about 1:3 and about 7:15.

32. The composition of claim 20, further comprising a photoactive molecule that changes its chemical structure after being exposed to a threshold level of EMR, such that it becomes opaque to one or more wavelength within the region of approximately 306 nm to approximately 320 nm.

33. A kit comprising the composition of claim 20, and further comprising a dosimeter that measures exposure to the one of said one or more predetermined wavelengths, and that provides an indication when said object has received a predetermined amount of EMR at the one of said one or more predetermined wavelengths.
